(19) United States
(12) Patent Application Publication
(10) Pub. No.: US 2004/0153152 A1
(43) Pub. Date: Aug. 5, 2004

(54) Tendon-replacement implant

(76) Inventors: Matthias Lowell, Nurnberg (DE); Klaus Landis, Heroldsberg (DE); Jurgen Henninger, Offenbach (DE)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/482,266

(22) PCT Filed: May 7, 2002
(86) PCT No.: PCT/EP02/05033

(30) Foreign Application Priority Data
Jun. 29, 2001 (DE) 101 31 699.2

(51) Int. Cl. A61F 2/08

(52) U.S. Cl. 623/13.12; 623/13.17

(57) ABSTRACT

The invention relates to an implant that replaces tendons, especially the interior cruciate ligament of the knee. The inventive implant comprises a first (10) and a second (12) base from spongy and/or cortical and/or cortical/spongy bone material of human or animal origin, linked via a flexible collagen strip (14) and adapted to be fixed in a corresponding implant seat by pressing.

The present invention relates to an implant for the replacement of tendons, in particular of the anterior cruciate ligament, in accordance with the preamble of claim 1 (cf. U.S. Pat. No. 6,090,998).

In a partial or total rupture of the cruciate ligament, an autologous or allogenic patellar bone block/patellar tendon/tibia bone block composite is usually implanted into the knee joint as a cruciate ligament replacement. Bores into which the bone blocks are implanted are introduced into the femur and into the tibia for the fixation of the bone blocks. The fixation of the implanted bone blocks in the bore passages in the femur or in the tibia takes place with appropriately dimensioned interference screws which can consist, for example, of metal (e.g. titanium) or of plastics (e.g. polyactide).

However, this method has disadvantages. The risk thus exists in the fixation with metallic interference screws that a bone block or a tendon section of the implant is injured or destroyed during the implanting. In addition, the interference screws remain in the implant bed for life and in diagnostic examinations such as X-ray examinations result in artifacts in the images obtained by the examination. After an implanting, a diagnosis on the healing success is therefore in particular only possible with reservations.

On the replacement of tendons, implants or parts thereof as well as the fixation elements can admittedly basically be used made of plastic. However, these are degraded over months, with the monomers or oligomers, which are created thereby, however, being able to result in damage to the implant or to the implant bed, i.e. to the bone, with corresponding complications such as a lysis of the bone.

An implant is known from U.S. Pat. No. 5,067,962 for the replacement of tendons which has a first and a second base body made of bone material which are connected via a collagen ligament. The base bodies are conical and are pinned in the implant bed by means of a steel pin.

On the replacement of tendons, implants or parts thereof as well as the fixation elements can admittedly basically be used made of plastic. However, these are degraded over months, with the monomers or oligomers, which are created thereby, however, being able to result in damage to the implant or to the implant bed, i.e. to the bone, with corresponding complications such as a lysis of the bone.

It is the object of the present invention to provide an implant for the replacement of tendons in whose implanting the risk of damage to the implant in the fixation is very reduced and in which artifact formation in diagnostic methods and damage to the implant bed after implanting is largely avoided.

The object is satisfied by an implant for the replacement of tendons having the features of claim 1.

The implant in accordance with the invention has two base bodies of bone material of human or animal origin which are connected via a flexible collagen ligament. The bone material can in each case be spongious and/or cortical and/or cortico-spongious material, with the two base bodies generally being able to be made of different bone materials. The base bodies can also be made as a composite of different ones of the named bone materials.

The flexible collagen ligament is fixed to the base bodies, with the fixation, depending on the choice of the starting material being able to be natural, for example when using a bone with an ongrown tendon, or artificial. In place, for example, by means of a biological adhesive or by means of mechanical fixation elements such as screws and pins. However, natural bone composites are preferred.

In accordance with the invention, the two base bodies are made by means of pressing into corresponding implant beds for the fixation.

This permits a particularly simple implanting of the implant since the surgeon only has to establish appropriate implant beds, e.g. by drilling, in order to then be able to anchor the base bodies in a stable manner by pressing into the implant bed without the use of fixation elements such as screws, plugs, pins or similar shaped bodies and without any further working. The base bodies implanted in this manner are then held in a press fit in the implant beds. The risk of an injury or damage to parts of the implant during the implanting is very much reduced by this implanting method.

The use of base bodies solely of bone material of human or animal origin and of a flexible collagen ligament moreover results in artifacts arising by the fixation of the implant are practically precluded in diagnostic methods producing images.

A particular advantage of the implant in accordance with the invention consists of its material not representing a foreign body due to the use only of materials of biological origin. The implant thereby contributes to the fixation and to the fusion between the implant and the implant bed in that it is converted into the body's own tissue during the healing.

it is converted into the body's own tissue during the healing.

The implant in accordance with the invention is admittedly also degraded over time, but no substances arise in this process which damage the implant bed; the implant is rather replaced by the body's own tissue over a medium-term period of time, without causing lysis in the implant bed by degradation products.

Advantageous embodiments and further developments of the invention are described in the description, in the drawings and in the dependent claims.

At least one of the base bodies preferably has a substantially cylindrical section and, adjoining it, a conical section at the implant end. This shape, on the one hand, permits a very simple establishment of the implant bed by introduction of a simple bore into the bone into which the base body is to be implanted. The conical section at the implant end, whose diameter there is smaller than that of the cylindrical section, on the other hand, permits a simple introduction of the base body into the implant bed. The length of the cylindrical section particularly preferably amounts to approximately 2/3 of the length of the base body and the length of the conical section amounts to approximately 1/3 of the length of the base body, with the length being understood as the extent of the base body in the direction of the cylinder axis. The size of the cone angle of the conical section is furthermore particularly preferably between approximately 2° and approximately 20°. An introduction of the base body into the implant bend is ensured by this only relatively low tapering without the risk of severe canting and of damage associated with it to the implant bed by the implant.

At least one of the base bodies furthermore preferably has peripheral sections with a corrugation which particularly preferably extends transversely to the later implanting direction. A particularly secure fit of the base body in the implant bed is ensured by such a corrugation. The corrugation particularly preferably has adjacent convex or linearly sawtooth-like recesses and ribs arranged between them, with the ribs running out in an edge in the region projecting from the base body. A particularly secure fit of the base body in the implant bed is in particular ensured by the edge tips which can be broken off in part by means of a ram on the implanting by hammering into the implant bed, then remain in the recesses of the corrugation and dig into the tissue of the implant bed and compact. In contrast, with a diameter of the implant which is somewhat too large and with an areal contact, the hammering-in forces can be so large that the risk of destroying the tissue exists, particularly at the contact position of the implanting tool, for example the ram, and the base body. If hollow spaces are intended to form in the region of the recesses between the tissue of the implant bed and the base body, they are first filled up with regrowing tissue such that a secure fit also results on the basis of a certain shape matching together with the edges which have dug in. A corrugation is particularly preferably formed at both base bodies.

manufactured particularly simply and moreover have a particularly good strength between the base body and the collagen ligament.

The material of the base body is preferably substantially spongious bone material which permits a particularly good ingrowth.

The implant in accordance with the invention preferably consists of processed, preserved and sterile bone material and, optionally, that is to the extent that the collagen ligament has not been gained by the demineralization of bone material, of tendon material of human origin, that is, it is a so-called allograft. An implant in accordance with the invention can particularly preferably, however, also consist of a processed, preserved and sterile bone material, and optionally tendon material, of animal origin and thus be a so-called xenograft. Bone material and tendon material of bovine, porcine and equine origin is particularly suitable in this process, since these can be gained without injury to the human body or removal of bone material or tendon material from the human body. Furthermore, the corresponding bone/tendon material can also be more stable than corresponding human material with a suitable choice.

A suitable allogenic or xenogenic bone material, and optionally tendon material, is preferably used as the material and is processed such that it is preserved, keepable and sterile such that it can be used in accordance with its intended purpose without the risk of infection.

The preservation of the bone material, and optionally of the tendon material, can take place, for example, by freeze drying. However, the bone material, and optionally the tendon material, is preferably produced by solvent dehydration of collagen bone material, and optionally tendon material, by means of an organic solvent miscible with water, e.g. methanol, ethanol, propanol, isopropanol, acetone, methyl ethyl ketone or mixtures of these solvents. Such a method for preservation and sterilization is described in the patent DE 29 06 650, whose content is included by this reference into the disclosure of the present application. Material manufactured in this manner has a structure in the histological appearance which very similar to the natural bone or to the natural tendon such that the desired properties of the collagen bone material, and optionally tendon material, remain. This is due to the fact that the method permits a dehydration and liberation right into the fine construction of the fibrillate of the collagen bone material, and optionally tendon material.

After the solvent dehydration, the bone material, and optionally the tendon material, can preferably be terminally sterilized, which can in particular take place by radiation with gamma rays or electron rays, but also by ethylene oxide or thermal processes.

Alternatively, the bone material, and optionally the tendon material, can, however, also be produced by aseptic processing of collagen bone material, and optionally tendon material, without terminal sterilization.

The implant is trimmed before sterilization; the base bodies can in particular be manufactured by machining with CNC controlled machines from bone/bone composites and/or bone/tendon composites or bone/tendon/bone composites. A reliable anchoring of the implant in the implant bed is ensured by the precise trimming achieved in this manner without mechanically damaging the implant or even the implant bed.

An implant in accordance with the invention is preferably used in a method for the replacement of tendons by implants, with at least one of the base bodies having a diameter which is between approximately 0.1 mm and approximately 3 mm larger in a direction perpendicular to the implanting direction than a corresponding implant bed opening into which the base body is implanted. It is particularly preferred in this process for the diameter of the base body to be approximately 0.5 mm larger than that of the implant body opening.

In addition to a use for the replacement of the cruciate ligament, a use is naturally generally also possible in the replacement of other tendons, such as the replacement of the Achilles tendon, thanks to differently sized dimensions of the base bodies and of the tendon portion of an implant in accordance with the invention.

A preferred embodiment of the invention will now be described by way of example with reference to the drawings. There are shown:

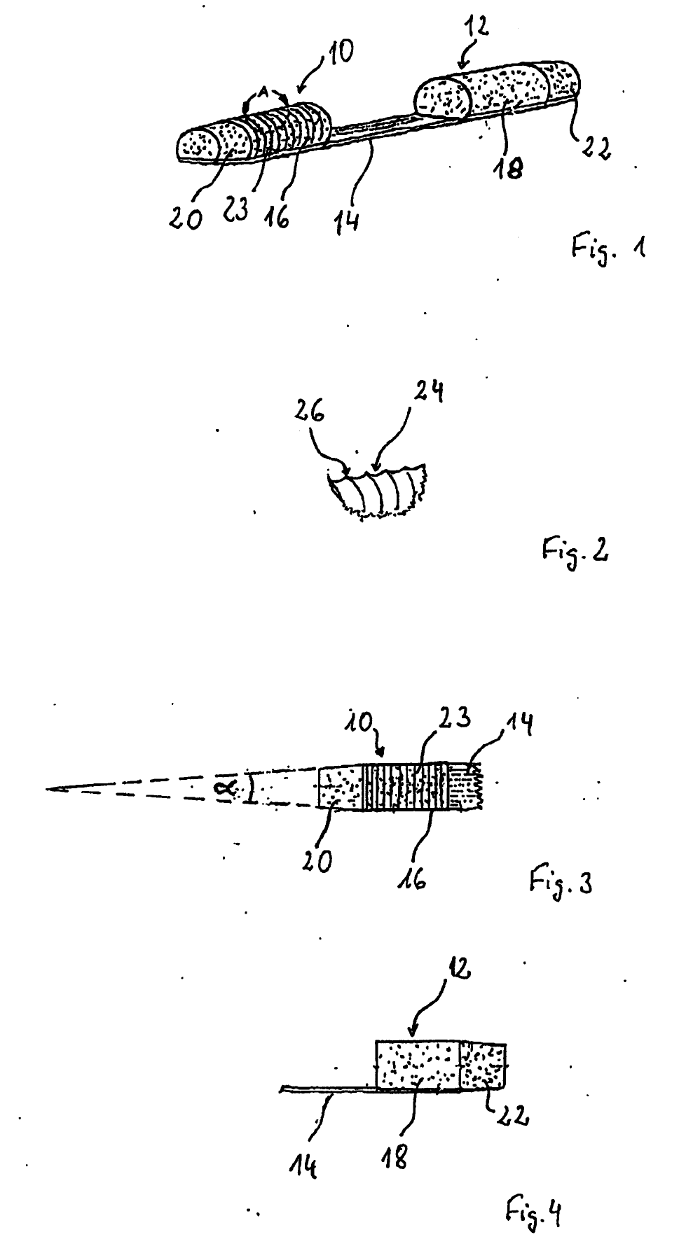

FIG. 1 a schematic perspective view of an implant for the replacement of a tendon in accordance with a preferred embodiment of the invention;

FIG. 2 a section from FIG. 1 which is marked there by A and which schematically shows the corrugation of the base body;

FIG. 3 a schematic plan view of the left hand base body in FIG. 1; and

FIG. 4 a schematic side view of the right hand base body in FIG. 1.

In FIG. 1, an implant in accordance with a preferred embodiment of the invention has a first base body 10 and a second base body 12, which are connected to one another by a flexible collagen ligament 14. The base bodies 10 and 12 each consist of a cortico-spongious bone material of porcine origin. The flexible collagen ligament 14 consists of a tendon material of allogenic or xenogenic origin.

As shown in FIGS. 1, 3 and 4, the bone blocks 10 and 12 are shaped substantially cylindrically to approximately 2/3 of their length and conically to approximately 1/3 of their length in each case toward the implant ends. The cone angle α, which is only shown in FIG. 3, but which is approximately of equal size for both base bodies, amounts to approximately 10°.

The flexible collagen ligament 14 is, as shown in FIGS. 1 and 4, connected over the total length of the corresponding cylindrical sections 16 and 18 to the respective base bodies 10 and 12.

The cylindrical section 16 of the base body 10 has, as shown in FIGS. 1 and 2, a corrugation 23 extending perpendicular to the cylinder axis, in which adjacent convex recesses 24 are separated by ribs 26. The profile of the ribs runs acutely to form an edge at their end extending away from the base body 10. A particularly secure fit of the implant in the implant bed is hereby ensured.

Generally, the external diameters of the implant, i.e. in particular the diameters of the cylindrical sections 16 and 18, are matched to the dimension of the implant bed. Possible dimensions of the described implant can be e.g. the following depending on the location of use:

The total length can vary between 75 and 200 mm, with the length of the free tendon lying between 20 and 100 mm and its width lying between approximately 4 and 30 mm. The diameter of the base bodies is not lower than the width of the tendon and can therefore also lie in the range between 34 mm. The length of the base bodies can lie between 15 and 40 mm.

### Reference Numeral List

10 first base body

12 second base body

14 flexible collagen ligament

16 cylindrical section

18 cylindrical section

20 conical section

22 conical section

23 corrugation

24 recess

26 rib

α cone angle

1. An implant for the replacement of tendons, in particular of the anterior cruciate ligament, with a first and a second base body (10, 12) made respectively of spongious and/or of cortical and/or of cortico-spongious bone material of human or animal origin which are connected via a flexible collagen ligament (14) and are designed for fixation by being pressed into a corresponding implant bed, characterized
in that at least one of the base bodies (10, 12) has a substantially cylindrical section (16, 18) and a conical section (20, 22) adjacent thereto at the implant end; and
in that said base body (10, 12) has peripheral sections with a corrugation (23) in order to permit an anchoring of the base body in an implant bed by means of a press fit.

2. An implant in accordance with claim 1, characterized in that the length of the cylindrical section (16, 18) has approximately 2/3 of the length of the base body and the length of the conical section has approximately 1/3 of the length of the base body.

3. An implant in accordance with one of the claims 1 or 2, characterized in that the size of the cone angle (α) of the conical section (20, 22) lies between approximately 2° and approximately 20°.

4. An implant in accordance with claim 1, characterized in that the corrugation (23) has adjacent convex recesses (24) and ribs (26) arranged between them, with the ribs running out in an edge in the region projecting from the base body.

5. An implant in accordance with any one of the preceding claims, characterized in that the flexible collagen ligament (14) is made by demineralization of human or animal bone material.

6. An implant in accordance with one of the claims 1 to 4, characterized in that the flexible collagen ligament (14) is formed from human or animal tendon material.

7 An implant in accordance with any one of the preceding claims, characterized in that the material of the base bodies (10, 12) is spongious bone material.

8. An implant in accordance with any one of the preceding claims, characterized in that it is obtained by processing with CNC controlled machines.
